SEQUENCE LISTING (16 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 330 aa, Homo sapiens
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 3 - protein, 270 aa, Homo sapiens
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 4 - protein, 330 aa, Homo sapiens
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 5 - protein, 578 aa, Homo sapiens [sequence omitted]

SEQ ID NO: 6 - protein, 331 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 7 - protein, 569 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 8 - DNA, 27 bp, Artificial Sequence
  Description of Artificial Sequence Synthetic primer
cagttccaattgtgtccaagtggatgt

SEQ ID NO: 9 - DNA, 29 bp, Artificial Sequence
  Description of Artificial Sequence Synthetic primer
catttcaaacaatctccaaggagatgatg

SEQ ID NO: 10 - DNA, 25 bp, Artificial Sequence
  Description of Artificial Sequence Synthetic probe
caaatgttctcaaagtgtggtcgct

SEQ ID NO: 11 - protein, 1231 aa, Homo sapiens
  MOD_RES (62)..(62): Val or Ile
MRLLAKIICLMLWAICVAEDCNELPPRRNTEILTGSWSDQTYPEGTQAIYKCRPGYRSLGNXIMVCRKGEWVALNPLXKCQKRPCGHPGDTPFGTFTLTGGNVFEYGVKAVYTCNEGYQLLGEINYXECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPXDYPDIKHGGLYHENMRRPYFPVAVGKYYSYYCDEHFETPSGSYWDHIHCTQDGWSPAVPCLRKCYFPYLENGYNXNXGRKFVQGKSIDVACHPGYALPKAQTTVTXMENGWSPTPRCIRVXXXXKSSIDIENGFISESQYTYALKEKAKYQCKLGYVTADGETSGSIXCGKDGWSAQPTCIKSCDIPVFMNARTKNDFTWFKLNDTLDYEXHDGYESNTGSTTGSXVCGYNGWSDLPICYEXECELPKIDVHLVPDRKKDQYKVGEVLKFSCKPGFTIVGPNSXQCYHFGLSPDLPICKEQVQSXGPPPELLNGNVKEKTKEEYGHSEVVEYYCNPRFLMKGPNKIQXVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEXITCKDGRWQSIPLCVEKIPCSQPPQIEHGTINSSRSSQEXYAXGTKLSYTCEGGFRISEENETTXYMGKWSSPPQCEGLPCKSPPXISHGVVAHMSDSYXXGEEVXYKXFEGFGIDGPAIAKCLGEKXSHPPSCIKTDCLSLPSFEXAIPMGEKKDXYKXGEQVTYXCATXYKMDGASNVTCINSRWTGRPTXRDTSCVXPPTVQNAYXVSRQMSKYPSGERVRYXCXSPYEMFGDEEVMCLNGNWTEPPQCKDSTGKCGPPPPIDNGXITSFPLSVYAPASSXEYQCQNLXXLEGNKRITCRNGQXSEPPKXLHPCVXSREIMENYNIALRXXAKQKXYXRTXESXXXVCKRGYRLSSXSHTLXTTCWDGKLEXXXXXXX

SEQ ID NO: 12 - protein, 330 aa, Homo sapiens
  MOD_RES (157)..(157): His or Tyr
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAXIXSRQMSKYPSGERVRYXCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESXEFVCKRGYRLSSRSHTLRTTCWDGKLEYPTCAKR

SEQ ID NO: 13 - protein, 270 aa, Homo sapiens
  MOD_RES (144)..(171): This region may encompass the peptide “ISAEKCGPPPPIDNGDITSFLLSVYAPG” or “S” wherein some positions may be absent
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTXXXXXXXXXXXXXXXXXXXXXXXXXXXXSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 14 - protein, 330 aa, Homo sapiens
  MOD_RES (71)..(71): His or Tyr
MLLLINVILTLWVSCANGQVKPCDFPDIKHGGLFHENMRRPYFPVAVGKYYSYYCDEHFETPSGSYWDYIXCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 15 - protein, 331 aa, Homo sapiens
  MOD_RES (306)..(306): Gly or Glu
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLXYNANTSVLSFQAVCREGIVEYPRCE

SEQ ID NO: 16 - protein, 569 aa, Homo sapiens
  MOD_RES (46)..(46): Pro or Ser
MLLLFSVILISWVSTVGGEGTLCDFPKIHHGFLYDEEDYNPFSQVXTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSXGEVKEIRKEEYGHNEVVEYDCNPNFIINGPKKIQCVDGEWTTLPTCVEQVKTCGYIPELEXGYVQPSVPPYQHGVSVEVNCRNEYAMIGNNMITCINGIWTELPMCVATHQLKRCKIAGVNIKTLLKLSGKEFNHNSRIXYRCSDIFRYRHSVCINGKWNPEXDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQXKWRNDGKXYAKTGDAVEFQCKFPHKAMISSPPFRAICQEGKFEYPICE

(19) United States
(12) Patent Application Publication
(10) Pub. No.: US 2014/0065610 A1
(43) Pub. Date: Mar. 6, 2014

(54) METHOD FOR PREDICTING CLINICAL BENEFIT IN THE TREATMENT OF NEURODEVELOPMENTAL, NEUROLOGICAL OR NEUROPSYCHIATRIC DISORDERS

(71) Applicants: Laurent Essioux, Attenschwiller (FR); Carsten Horn, Basel (CH); Johann Karl, Peissenberg (DE); Peter Kastner, Muenchen (DE); Fabian Model, Penzberg (DE); Giuseppe Palermo, Basel (CH); Christina Rabe, Bad Heilbrunn (DE); Daniel Umbricht, Zuerich (CH); Gabriel Vargas, Thousand Oaks, CA (US); Anne Vogt, Loerrach (DE)

(72) Inventors: Laurent Essioux, Attenschwiller (FR); Carsten Horn, Basel (CH); Johann Karl, Peissenberg (DE); Peter Kastner, Muenchen (DE); Fabian Model, Penzberg (DE); Giuseppe Palermo, Basel (CH); Christina Rabe, Bad Heilbrunn (DE); Daniel Umbricht, Zuerich (CH); Gabriel Vargas, Thousand Oaks, CA (US); Anne Vogt, Loerrach (DE)

(21) Appl. No.: 13/590,211

(22) Filed: Aug. 21, 2012

(30) Foreign Application Priority Data
Aug. 26, 2011 (EP) 11179058.0
Apr. 13, 2012 (EP) 12164017.1

(51) Int. Cl. G01N 33/68; C12Q 1/68

(52) U.S. Cl. CPC G01N 33/6896; C12Q 1/6883
USPC 435/6.11; 435/7.4

(57) ABSTRACT

An in vitro method of predicting whether a patient, having a neurodevelopmental, neurological or neuropsychiatric disorder, will derive a clinical benefit if treated with a glycine reuptake inhibitor (GRI), via determination of the protein concentration of one, two, three, four five or six members of the complement factor H family or a mixture or a combination thereof and comparison against a representative value, wherein a higher value of the protein concentration in the patient's sample against the representative value is indicative of a patient whom will derive clinical benefit from treatment with GRI.

### PRIORITY TO RELATED APPLICATION(S)

This application claims the benefit of European Patent Application No. 11179058.0, filed Aug. 26, 2011, which is hereby incorporated by reference in its entirety.

### FIELD OF THE INVENTION

The present invention relates to methods which are predictive for the clinical benefit for the treatment of patients having neurodevelopmental, neurological or neuropsychiatric disorders with a glycine reuptake or other compound inhibitor, targeting the glutamatergic pathway.

### BACKGROUND OF THE INVENTION

Glycine Reuptake Inhibitors, which targets GlyT1, (GRI) are a novel class of compounds that are thought to enhance NMDA receptor (NMDA-R) mediated transmission by elevating extracellular concentrations of glycine. Evidence from studies in healthy individuals, psychotic patients and animals as well as from genetic analysis has accumulated over the past 15 years of the involvement of NMDA receptor (NMDA-R) hypo-function in the pathophysiology of neurodevelopmental, neurological or neuropsychiatric disorders.

In CNS, glycine has two major roles for controlling sensory and higher brain function: it is an inhibitory neurotransmitter in glycinergic neurons and an exitatory neurotransmitter as a co-agonist with glutamate of the glutamatergic transmission of the NMDA receptor.

Glutamate is the main excitatory neurotransmitter in the brain and activates NMDA and non-NMDA receptors (ligand-gated ion channels, i.e. AMPA, kainite and metabotropic receptors, i.e. mGluR 1-8). NMDA receptors are located on different neurons, such as glutamatergic, dopaminergic and GABAergic. NMDA receptors can therefore directly affect Glutamate, GABA and Dopamine.

NMDA receptors are ligands gated ion channels meaning they require for their activation the binding of both glutamate and glycine. Glycine acts as modulator of glutamate: it increases the potency of glutamate thus enhancing its effect on activating the receptor. In the developing brain and, to a lesser extent in the mature brain, NMDA receptors play a crucial role in synaptogenesis and in promoting synaptic maintenance and stabilization (via increased synaptic plasticity). NMDA receptors are therefore involved in several brain functions both in mature and in developing brain.

A dysfunction in glutamatergic neurotransmission is involved in the pathophysiology of mood disorders and schizophrenia. Thus, it is likely that the NMDA receptor glycine-modulatory site is a therapeutic target for improving cognition and reducing negative symptoms in schizophrenia.

As glycine is an obligatory co-agonist at the NMDA-R complex, one strategy to enhance NMDA-R mediated neurotransmission is to elevate extracellular concentrations of glycine in the local microenvironment of NMDA receptors. Glycine elevation can be achieved by inhibition of GRI, which is responsible for glycine removal from the synaptic cleft. Possible advantages over the existing neurological and neuropsychiatric therapies include the potential of glycine reuptake inhibitors in having good efficacy, as well as an improved tolerability profile for the treatment of negative and positive symptoms in schizophrenia, bipolar disorders, substance dependence (alcohol, cocaine), autism or obsessive compulsive disorders (OCD).

GlyT1 belongs to the superfamily of neurotransmitter transporters, like SERT, NET or DAT. GlyT1 surrounds the glutamatergic synapse in the forebrain. It maintains extracellular glycine concentration in the synapse below the saturating level at its binding site on NMDA receptor's NR1 site. In the caudal brain region and spinal cord, GlyT1 controls both glutamatergic (via NMDA receptors) and glycinergic transmission.

It is known that glycine reuptake inhibitors may be used for the treatment of neurodevelopmental, neurological or neuropsychiatric disorders, such as schizophrenia. Other indications associated with glutamatergic transmission are bipolar disorders, substance dependence (alcohol, cocaine), autism and obsessive compulsive disorders (OCD).

It has long been acknowledged that there is a need to develop methods of individualizing treatment of CNS diseases such as schizophrenia, bipolar disorders, substance dependence (alcohol, cocaine), autism and obsessive compulsive disorders (OCD).

With the development of targeted disease treatments, there is a particular interest in methodologies which could provide a molecular profile of the target, (i.e. those that are predictive for clinical benefit).

Therefore, it is an object of the present invention to provide an in vitro method to predict clinical benefit to a compound, targeting the glutamatergic pathway, e.g. to identify individuals who will derive clinical benefit to treatment with a compound, targeting the glutamatergic pathway in related diseases, which are schizophrenia, bipolar disorders, substance dependence (alcohol, cocaine), autism and obsessive compulsive disorders (OCD).

For this purpose, a predictive marker present in the circulation which is detectable in body fluids (e.g. blood, serum or plasma) has to be found.

In order to be of clinical utility, a predictive marker should be able to discriminate those individuals who will derive clinical benefit from those individuals who will not derive clinical benefit from treatment with a compound, targeting the glutamatergic pathway. The predictive sensitivity or specificity of a test is best assessed by its receiver-operating characteristics.

Whole blood, serum or plasma are the most widely used sources of sample in clinical routine. The identification of a predictive marker that would aid in the identification of individuals who are likely to respond to treatment with a compound, targeting the glutamatergic pathway in schizophrenia and other diseases could lead to a method that would greatly aid in the treatment and in the management of these diseases. Therefore, an urgent clinical need exists to improve the treatment of diseases, targeting the glutamatergic pathway, for example GRI related diseases.

The object of the present invention is further to investigate whether a biochemical marker can be identified which may be used in predicting response to a compound, targeting the glutamatergic pathway, for example to GRI in vitro in a body fluid sample.

Surprisingly it has been found that an in vitro determination of the concentration of protein CFH family in a sample allows the prediction of a clinical benefit from the treatment with GRI for patients with neurodevelopmental, neurological or neuropsychiatric disorders.

### SUMMARY OF THE INVENTION

The invention discloses the use of the proteins of the complement factor H family as predictive markers for clinical benefit for patients which are treated with a compound, targeting the glutamatergic pathway, for example with a glycine reuptake inhibitor. For example, and more preferably, the glycine reuptake inhibitor is [4-(3-fluoro-5-trifluoromethyl-pyridin-2-yl)-piperazin-1-yl]-[5-methanesulfonyl-2-((S)-2,2,2-trifluoro-1-methyl-ethoxy)-phenyl]-methanone. Furthermore, it especially relates to a method for predicting drug response from a sample, derived from an individual by measuring proteins of complement factor H family in said sample in vitro.

Complement factor H family is defined as CFH proteins selected from the group consisting of CFH, CFHR1, CFHR2, CFHR3, CFHR4A, CFHR4B and CFHR5. In a preferred enablement, the complement factor H family may be a mixture of two or more of said group of CFH proteins.

### DESCRIPTION OF THE FIGURES

FIG. 1: Histogram of CFHR1 concentrations in the Phase II serum baseline samples. The three curves show the probability density estimates from a clustering algorithm (Mixture of Gaussians). The clustering analysis assumed 3 different concentration groups based on the genetic status of CFHR1 expression. 2 natural cut-offs can be identified using the CFHR1 ECLIA immuno assay: at 8 μg/ml and at 28 μg/ml. The 28 μg/ml cut-off (marked with an arrow) separates the patients with CFHR1 heterozygous or homozygous deletion from those carrying no deletion and was used for patient stratification.

FIG. 2: Change of PANSS negative symptom factor score from baseline in all patients (FIG. 2(a)) and in CFHR1-low patients (FIG. 2(b)) and CFHR1-high patients (FIG. 2(c)) using natural cutoff for CFHR1. Patients with serum concentration ≦28 ug/ml were classified as CFHR1-low and patients with serum concentration >28 ug/ml were classified as CFHR1-high. Estimates of expected response and standard error bars from MMRM analysis. Dash-dot line and circle Placebo; solid line and squares 10 mg GRI; solid line and upward-pointing triangles 30 mg GRI; dashed line and diamonds 60 mg GRI.

FIG. 3: Response rates of dichotomized PANSS negative symptom factor score (response defined as ≧20% drop from baseline) at week 8. Shaded background bars are response rates in overall per protocol population. Solid grey bars are response rates in CFHR1 stratified subpopulations using natural cut-off for CFHR1.

FIG. 4: Response rates of dichotomized CGI-I negative symptoms rating score (response defined as “much improved” or “very much improved”) at week 8. Shaded background bars are response rates in overall per protocol population. Solid grey bars are response rates in CFHR1 stratified subpopulations using natural cut-off for CFHR1.

FIG. 5: Histogram of the CFHR1/CFH ratio distribution in the Phase II serum baseline samples. The three curves show the probability density estimates from a clustering algorithm (Mixture of Gaussians). The clustering analysis assumed 3 different concentration groups based on the genetic status of CFHR1 expression. 2 natural cut-offs can be identified using the CFHR1/CFH ratio (measured by the ECLIA assays): 0.01 and 0.08. The samples with a CFHR1/CFH ratio below 0.01 are indicative of CFHR1 homozygous deletion, samples with a CFHR1/CFH ratio above 0.01 and below 0.08 are indicative of CFHR1 heterozygous deletion, and samples with a CFHR1/CFH ratio above 0.08 are indicative of no CFHR1 deletion

FIG. 6: Response rates of dichotomized CGI-I negative symptoms rating score (response defined as “much improved” or “very much improved”) at week 8. Shaded background bars are response rates in overall per protocol population. Solid grey bars are response rates in CFHR1 stratified subpopulations using CFHR1/CFH ratio, with L (low) corresponding to CFHR1/CFH ratio below 0.01, M (medium) corresponding to CFHR1/CFH ratio above 0.01 and below 0.08 and with H (high) corresponding to CFHR1/CFH ratio above 0.08.

FIG. 7: Genetic status for CFHR1 gene versus CFHR1 protein concentration using the RBM assay (recognizing CFHR1:CFH in a ration 10:1)

FIG. 8: Allelic status for SNP rs7542235 versus CFHR1 protein concentration using the RBM assay (recognizing CFHR1:CFH in a ration 10:1)

FIG. 9: Response rates of dichotomized CGI-I negative symptoms rating score (response defined as “much improved” or “very much improved”) at week 8. Shaded background bars are response rates in overall per protocol population from those who had consented for DNA sample. Solid grey bars are response rates in CFHR1 stratified subpopulations using genetic status of CFHR1 allele.

### DESCRIPTION OF THE SEQUENCES (SEQ IDS)

Seq. Id. No. 1: shows the protein sequence of human complement factor H.

Seq. Id. No. 2: shows the protein sequence of human complement factor H related protein CFHR1.

Seq. Id. No. 3: shows the protein sequence of human complement factor H related protein CFHR2.

Seq. Id. No. 4: shows the protein sequence of human complement factor H related protein CFHR3.

Seq. Id. No. 5: shows the protein sequence of human complement factor H related protein CFHR4A.

Seq. Id. No. 6: shows the protein sequence of human complement factor H related protein CFHR4B

Seq. Id. No. 7: shows the protein sequence of human complement factor H related protein CFHR5.

Seq. Id. No. 8 shows the oligonucleotide sequence for the forward primer used in the customized CFHR1 copy number assay

Seq. Id. No. 9 shows the oligonucleotide sequence for the reverse primer used in the customized CFHR1 copy number assay

Seq. Id. No. 10 shows the oligonucleotide sequence for the probe used in the customized CFHR1 copy number assay

### DETAILED DESCRIPTION OF THE INVENTION

The invention discloses the use of the proteins of the complement factor H family as predictive markers for clinical benefit for patients which are treated with a compound, targeting the glutamatergic pathway, for example with a glycine reuptake inhibitor. More specifically, [4-(3-fluoro-5-trifluoromethyl-pyridin-2-yl)-piperazin-1-yl]-[5-methanesulfonyl-2-((S)-2,2,2-trifluoro-1-methyl-ethoxy)-phenyl]-methanone. Furthermore, it especially relates to a method for predicting drug response from a sample, derived from an individual by measuring proteins of complement factor H family in said sample in vitro.

Complement factor H family is defined as CFH proteins selected from the group consisting of CFH, CFHR1, CFHR2, CFHR3, CFHR4A, CFHR4B and CFHR5. In a preferred enablement, the complement factor H family may be a mixture of two or more of said group of CFH proteins.

In this context it was found that determining the protein concentration of complement factor H family members in the blood, serum or plasma sample of a patient is indicative for a patient who will derive clinical benefit from the treatment. For this purpose the protein concentration of complement factor H family members are compared to a value representative of the protein concentration of complement factor H family members of a population of patients deriving no clinical benefit from the treatment, wherein a higher protein concentration of complement factor H family members in the blood, serum or plasma sample of the patient is indicative for a patient who will derive clinical benefit from the treatment.

A new in vitro method of predicting the response of a patient with neurodevelopmental, neurological or neuropsychiatric disorders is provided, wherein such patient is treated with a compound, targeting the glutamatergic pathway, for example with GRI, which method comprises:

determining the protein concentration of complement factor H family member(s) in the blood, serum or plasma sample of a patient and

comparing the protein concentration of complement factor H family member(s) to a value representative of the protein concentration of complement factor H family member(s) of a population of patients deriving no clinical benefit from the treatment, wherein a higher protein concentration of complement factor H family member(s) in the blood, serum or plasma sample of the patient is indicative for a patient who will derive clinical benefit from the treatment.

In a further embodiment the present invention relates to the use of the protein complement factor H family member(s) in the in vitro assessment of certain CNS diseases, such as schizophrenia, in a sample, wherein a concentration of protein complement factor H family member(s) above a reference concentration is indicative for clinical benefit due to drug treatment of said diseases.

The complement factor H was selected from a panel of 189 proteins (Rules Based Medicine, Human Discovery Map v 1.0). Baseline and treatment serum samples from schizophrenia patients treated with GRI were analyzed on the Luminex based multiplex ELISA Human DiscoveryMap® v 1.0.

DiscoveryMAP® is a comprehensive quantitative immunoassay test, containing 189 protein analytes. It represents the culmination of 10 years of assay development for cytokines, chemokines, metabolic markers, hormones, growth factors, tissue remodeling proteins, angiogenesis markers, acute phase reactants, cancer markers, kidney toxicity markers, CNS biomarkers and other important serum proteins.

Complement factor H was found superior to the other 189 protein analytes in view of the following advantages:

complement factor H shows a strong discrimination between responders and non-responders in the treatment group

complement factor H shows no prognostic effect in the placebo group;

complement factor H serum concentration is not influenced by treatment with GRI within 8 weeks

complement factor H serum concentration is stable over time.

Complement factor H, also known as factor H, is a sialic acid containing glycoprotein that plays an integral role in the regulation of the complement-mediated immune system that is involved in microbial defense, immune complex processing, programmed cell death and age-related macula degeneration. Complement factor H is the best characterized member of the complement factor H protein family. The complement factor H family consists of the following members: complement factor H(CFH), complement factor H-related protein 1 (CFHR1), complement factor H-related protein 2 (CFHR2), complement factor H-related protein 3 (CFHR3), complement factor H-related protein 4 with isoforms 4A and 4B (CFHR4A and CFHR4B), complement factor H-related protein 5 (CFHR5). The complement factor H-related proteins are encoded downstream of the complement factor H gene and share a high concentration of homology with subdomains of complement factor H. Complement factor H related proteins share also functional similarities [4].

The complement system consists of ˜40 proteins that are present in body fluids or on cell and tissue surfaces and is activated in a cascade-like manner by three major pathways [1]. The alternative pathway is activated continuously at a low rate by the spontaneous hydrolysis of the central component C3, the lectin pathway is initiated by mannose binding lectin or ficolins that recognize microbial carbohydrates and the classical pathway is activated by binding of Clq to antigen bound immunoglobulins. Enzymatic steps generate active fragments of complement components and trigger further amplification. The three pathways merge at the concentration of C3, which on activation, is cleaved into C3a and C3b.

Complement factor H protects host cells from injury resulting from unrestrained complement activation. Complement factor H regulates complement activation on self cells by possessing both cofactor activity for the Factor I mediated C3b cleavage, and decay accelerating activity against the alternative pathway C3 convertase, C3bBb.

Complement factor H protects self cells from complement activation but not bacteria/viruses. Due to the central role that Complement factor H plays in the regulation of complement, there are many clinical implications arising from aberrant CFH activity. Mutations in the Complement factor H gene are associated with severe and diverse diseases including the rare renal disorders hemolytic uremic syndrome (HUS) and membranoproliferative glomerulonephritis (MPGN) also termed dense deposit disease (DDD), membranoproliferative glomuleronephritis type II or dense deposit disease, as well as the more frequent retinal disease age related macular degeneration (AMD). In addition to its complement regulatory activities, complement factor H has multiple physiological activities and 1) acts as an extracellular matrix component, 2) binds to cellular receptors of the integrin type, and 3) interacts with a wide selection of ligands, such as the C-reactive protein, thrombospondin, bone sialoprotein, osteopontin, and heparin.

Complement factor H related protein 1 (CFHR1) is a 43 kDa, secreted member of the factor H family of glycoproteins. It is produced by hepatocytes and circulates as two differentially glycosylated isoforms (37 kDa and 43 kDa). Mature human CFHR1 is 312 aa in length. It contains five approximately 60 aa SCRs (short consensus repeats/CCPs/SUSHI repeats) that basically constitute the entire molecule. CFHR1 may play a role in lipoprotein complexes that bind LPS to neutrophils. There is no reported rodent counterpart to CFHR1. Over aa 19143 of the CFHR1 precursor, human CFHR2 and CFHR5 show 98% and 86% aa identity, respectively.

### As Used Herein

A neurodevelopmental disorder is an impairment of the growth and development of the brain or central nervous system. A narrower use of the term refers to a disorder of brain function that affects emotion, learning ability and memory, and social competence, skills and behaviors and that unfolds as the individual grows. Disorders considered to be neurodevelopmental in origin or to have neurodevelopmental consequences when they occur in infancy and childhood include autism and autism spectrum disorders such as Asperger syndrome, traumatic brain injury (including congenital injuries such as those that cause cerebral palsy, communication, speech and language disorders, genetic disorders such as fragile-X syndrome and Down syndrome. Neurodevelopmental disorders are associated with widely varying degrees of mental, emotional, physical and economic burden to individuals, families and society in general.

A neurological disorder is a disorder of the body's nervous system. Structural, biochemical or electrical abnormalities in the brain, spinal cord, or in the nerves leading to or from them, can result in symptoms such as paralysis, muscle weakness, poor coordination, loss of sensation, seizures, confusion, pain, apathy and altered states of consciousness. There are many recognized neurological disorders, some relatively common, but many rare. The World Health Organization estimated in 2006 that neurological disorders and their sequelae affect as many as one billion people worldwide, and identified health inequalities and social stigma/discrimination as major factors contributing to the associated disability and suffering.

Neuropsychiatry is the branch of medicine dealing with mental disorders attributable to diseases of the nervous system and it is also closely related to the field of psychiatry and behavioral neurology, which is a subspecialty of neurology that addresses clinical problems of cognition and/or behavior caused by brain injury or brain disease.

Neurodevelopmental, neurological or neuropsychiatric disorders include schizophrenia, bipolar disorders, substance dependence (alcohol, cocaine), autism and obsessive compulsive disorders (OCD), which are the most preferred indications with respect to the present invention.

Schizophrenia is a severe mental disorder typically appearing in late adolescence or early adulthood with a word-wide prevalence of approximately 1% of the adult population which has enormous social and economic impact. The criteria of the Association of European Psychiatrists (ICD) and the American Psychiatric Association (DSM) for the diagnosis of schizophrenia require that two or more characteristic symptoms be present: delusions, hallucinations, disorganized speech, grossly disorganized or catatonic behavior, or negative symptoms (alogia, affective flattening, lack of motivation, anhedonia), and that other requirements, such as excluding affective disorders, and the presence of impaired function, be present. As a group, people with schizophrenia have functional impairments that may begin in childhood, continue throughout adult life and make most patients unable to maintain normal employment or otherwise have normal social function. [2,3]. They also have a shortened lifespan compared to the general population, and suffer from an increased prevalence of a wide variety of other neuropsychiatric syndromes, including serious, substance abuse, obsessive-compulsive symptoms and abnormal involuntary movements prior to antipsychotic treatment. Schizophrenia is also associated with a wide range of cognitive impairments, the severity of which limits their function, even when psychotic symptoms are well controlled.

The term “biomarker or marker” means a substance used as an indicator of a biological state, i.e. of biological processes or pharmacologic response to a therapeutic interaction. A biomarker or marker indicates a change in expression or state of a protein that correlates with the risk or progression of a disease, or with the susceptibility of the disease to a given treatment.

The term “gene” means a piece of DNA in the host organism.

Gene expression is the process in which information from a gene is used in the synthesis of a functional gene product, e.g. a protein. The term “expression concentration” means the concentration at which a particular gene is expressed within a cell, tissue or organism. The term “expression concentration” in context with proteins reflects the amount of a particular protein present in a cell at a certain time.

The term “protein” means a functional gene product that has been synthesized from the gene.

The term “a value representative of a protein concentration of complement factor H family members of a population of patients deriving no clinical benefit from the treatment” refers to an estimate of a mean expression concentration of a population of patients who do not derive a clinical benefit from the treatment. Clinical benefit was defined as having a measurable response compared to placebo after 8 weeks.

The term “sample” or “test sample” as used herein refers to a biological sample obtained from an individual for the purpose of evaluation in vitro. In the methods of the present invention, the sample or patient sample may comprise in an embodiment of the present invention any body fluid. Preferred samples are body fluids, such as blood, serum or plasma, with serum or plasma being most preferred.

The term “mixture” refers to a protein mixture made up by two or more proteins that are simultaneously detected by one protein assay, e.g. a sandwich assay. The simultaneous detection can be due to similar epitopes being present on the proteins detected by the antibodies used in the sandwich assay.

The term “combination” relates to independent measurements of two or more protein analytes measured out of a larger group of proteins, where the order does not matter. The independent measurement results are combined mathematically, e.g. by calculating a ratio of two measurement results.

Protein concentrations of complement factor H family member(s), particularly soluble forms of protein concentrations of complement factor H family member(s) (CFH, CFHR1, CFHR2, CFHR3, CFHR4A, CFHR4B and CFHR5 or mixtures thereof), are determined in vitro in an appropriate sample. Preferably, the sample is derived from a human subject, e.g. a schizophrenia patient or a person in risk of schizophrenia or a person suspected of having schizophrenia. Protein concentrations of complement factor H family member(s) are determined in a blood, serum or plasma sample.

The invention comprises a method of predicting a response for patients, having neurodevelopmental, neurological or neuropsychiatric disorders, if treated with a compound, targeting the glutamatergic pathway, comprising the steps

i) determining the protein concentration of one, two, three, four five or six members of the complement factor H family or a mixture or a combination thereof in a sample of a patient,
ii) comparing the protein concentration determined in step i) to a value representative of the protein concentration of one, two three, four, five or six members of complement factor H family in patients, having neurodevelopmental, neurological or neuropsychiatric disorders,
iii) wherein a higher protein concentration of one, two three, four five or six members from complement factor H family is detected in the sample of the patient having neurodevelopmental, neurological or neuropsychiatric disorders in comparison to the value representative in step ii), is indicative for a patient who will derive clinical benefit from this treatment and
iv) selecting this treatment for patients having neurodevelopmental, neurological or neuropsychiatric disorders.

More specifically, the invention comprises an in vitro method of predicting a response for patients, having neurodevelopmental, neurological or neuropsychiatric disorders, if treated with a glycine reuptake inhibitor (GRI), comprising the steps

i) determining the protein concentration of one, two, three, four five or six members of the complement factor H family or a mixture or a combination thereof in a sample of a patient,
ii) comparing the protein concentration determined in step i) to a value representative of the protein concentration of one, two three, four, five or six members of complement factor H family in patients, having neurodevelopmental, neurological or neuropsychiatric disorders,
iii) wherein a higher protein concentration of one, two three, four five or six members from complement factor H family is detected in the sample of the patient having neurodevelopmental, neurological or neuropsychiatric disorders in comparison to the value representative in step ii), is indicative for a patient who will derive clinical benefit from treatment with GRI, and
iv) selecting GRI treatment for patients having neurodevelopmental, neurological or neuropsychiatric disorders.

The complement factor H family members include proteins of complement factor H (CFH), complement factor H related protein 1 (CFHR1), complement factor H related protein 2 (CFHR2), complement factor H related protein 3 (CFHR3), complement factor H related protein 4A CFHR4A), complement factor H related protein 4B (CFHR4B), and complement factor H related protein 5 (CFHR5).

One embodiment of the invention are complement factor H family members as mentioned above or a mixture or a combination thereof.

One embodiment of the invention is that the complement factor H family members are complement factor H related protein 1, or a mixture of complement factor H related protein 1 and complement factor H.

One further embodiment of the invention is an in vitro method, wherein the complement factor H family member is complement factor H related protein 1.

One further embodiment of the invention is an in vitro method, wherein the complement factor H family member is a combination of complement factor H related protein 1 and complement factor H

One particularly preferred embodiment of the invention is an in vitro method, wherein the complement factor H family member is the ratio of complement factor H related protein 1 and complement factor H.

One embodiment of the invention is further an in vitro method, wherein the complement factor H family member is a combination of complement factor H related protein 3 and complement factor H.

One embodiment of the invention is further an in vitro method, wherein the complement factor H family member is the ratio of complement factor H related protein 3 and complement factor H.

One embodiment of the invention is further an in vitro method, wherein the complement factor H family member is a combination of either complement factor H related protein 3 or complement factor H related protein 1 and one of the following complement factors: complement factor H related protein 2, complement factor H related protein 4A, complement factor H related protein 4B, complement factor H related protein 5 and complement factor H.

One embodiment of the invention is further an in vitro method, wherein the complement factor H family member is the ratio of either complement factor H related protein 3 or complement factor H related protein 1 and one of the following complement factors: complement factor H related protein 2, complement factor H related protein 4A, complement factor H related protein 4B, complement factor H related protein 5 and complement factor H.

The method of predicting a response for patients, having neurodevelopmental, neurological or neuropsychiatric disorders include negative or positive symptoms of schizophrenia, bipolar disorder, substance dependence, autism and compulsive disorders. One embodiment of the invention is a method where the patient is affected with schizoaffective disorder.

One further embodiment of the invention is that the protein concentration of individual members of the complement factor H family or a mixture or a combination thereof are determined by ELISA based technology.

The invention further comprises an in vitro method of predicting a response for patients, having neurodevelopmental, neurological or neuropsychiatric disorders, if treated with a compound, targeting the glutamatergic pathway which may be a glycine reuptake inhibitor (GRI), wherein the protein concentration of individual members of the complement factor H family or a mixture or a combination thereof are determined by measuring genetic variants of complement factor H family members [5].

The invention further comprises an in vitro method of predicting a response for patients, having neurodevelopmental, neurological or neuropsychiatric disorders, if treated with a glycine reuptake inhibitor (GRI) wherein the protein concentration of CFHR1 is determined by measuring of genetic variants of CFHR1, either via measurement of copy number variations of CFHR1 or by measurement of a SNP as a proxy for the deletion.

A further embodiment of the invention is the use of an antibody specifically binding to a protein of the complement factor H family.

The in vitro method comprises the use of a GRI, which is [4-(3-fluoro-5-trifluormethyl-pyridin-2-yl)-piperazin-1-yl]-[5-methanesulfonyl-2-[[(2S)-1,1,1-trifluoropropan-2-yl]oxy]phenyl]methanone.

A further embodiment of the invention is a complement factor H family member for use as a predictive marker for patients who are treated with a compound targeting the glutamatergic pathway, for example with a glycine reuptake inhibitor (GRI).

A further embodiment of the invention is the use of complement factor H family member as a predictive marker for clinical benefit for patients, having neurodevelopmental, neurological or neuropsychiatric disorders, if treated with a compound targeting the glutamatergic pathway, for example with a glycine reuptake inhibitor (GRI).

A further embodiment of the invention is a complement factor H family member as a predictive marker for clinical benefit for patients, having neurodevelopmental, neurological or neuropsychiatric disorders, if treated with a compound targeting the glutamatergic pathway, for example with a glycine reuptake inhibitor (GRI).

The term “reference sample” as used herein refers to a biological sample provided from a reference group of patients deriving no clinical benefit from the treatment for the purpose of evaluation in vitro. The term “reference concentration” as used herein refers to a value established in a reference group of patients deriving no clinical benefit from the treatment.

It is known to a person skilled in the art that the measurement results of step (i) according to the method(s) of the present invention will be compared to a reference concentration. Such reference concentration can be determined using a negative reference sample, a positive reference sample, or a mixed reference sample comprising one or more than one of these types of controls.

The expression “comparing the concentration determined to a reference concentration” is merely used to further illustrate what is obvious to the skilled artisan anyway. A reference concentration is established in a control sample. The control sample may be an internal or an external control sample. In one embodiment an internal control sample is used, i.e. the marker concentration(s) is (are) assessed in the test sample as well as in one or more other sample(s) taken from the same subject to determine if there are any changes in the concentration(s) of said marker(s). In another embodiment an external control sample is used. For an external control sample the presence or amount of a marker in a sample derived from the individual is compared to its presence or amount in an individual known to suffer from, or known to be at risk of, a given condition; or an individual known to be free of a given condition, i.e., “normal individual”. For example, a marker concentration in a patient sample can be compared to a concentration known to be associated with a specific course of neurodevelopmental, neurological or neuropsychiatric disorders.

Usually the sample's marker concentration is directly or indirectly correlated with a diagnosis and the marker concentration is e.g. used to determine whether an individual is at risk for neurodevelopmental, neurological or neuropsychiatric disorders.

Alternatively, the sample's marker concentration can e.g. be compared to a marker concentration known to be associated with a response to therapy in schizophrenia patients, the diagnosis of schizophrenia, the guidance for selecting an appropriate drug to schizophrenia, in judging the risk of disease progression, or in the follow-up of schizophrenia patients. Depending on the intended diagnostic use an appropriate control sample is chosen and a control or reference value for the marker established therein. It will be appreciated by the skilled artisan that such control sample in one embodiment is obtained from a reference population that is age-matched and free of confounding diseases. As also clear to the skilled artisan, the absolute marker values established in a control sample will be dependent on the assay used. Preferably samples from 100 well-characterized individuals from the appropriate reference population are used to establish a reference value. Also preferred the reference population may be chosen to consist of 20, 30, 50, 200, 500 or 1000 individuals. Schizophrenia patients who derive no clinical benefit from GRI treatment represent a preferred reference population for establishing a reference value.

Schizophrenia patients who derive no clinical benefit from GRI treatment can be defined as patients who show no or only minimal improvement in their symptoms after treatment. In an embodiment, no or minimal improvement in symptoms is defined as less than 20% change in the Positive and Negative Symptom Scale (PANSS). In another embodiment, no or minimal improvement in symptoms is defined as worse symptoms, no change or minimally improved symptoms according to the Clinical Global Impression scale (CGI).

Schizophrenia patients with a specific genotype of CFH family genes represent another preferred reference population for establishing a control value. In an embodiment, schizophrenia patients with a homozygous deletion of the CFHR1 and CFHR3 genes represent a preferred reference population for establishing a reference value. In another embodiment, schizophrenia patients with a homozygous or heterozygous deletion of the CFHR1 and CFHR3 genes represent a preferred reference population for establishing a reference value.

The term “measurement”, “measuring” or “determining” can be qualitative, semi-quantitative or quantitative. In the present invention a protein of Complement factor H family members or a mixture thereof is measured in a body fluid sample. In a preferred embodiment the measurement is a semi-quantitative measurement, i.e. it is determined whether the concentration of a protein of Complement factor H family members or a mixture thereof is above or below a cut-off value.

The values for protein of Complement factor H family members or a mixture thereof as determined in a control group or a control population are for example used to establish a cut-off value or a reference range. A value above such cut-off value is considered as indicative for the prediction of clinical benefit in the treatment of the neurodevelopmental, neurological or neuropsychiatric disorders.

In an embodiment a fixed cut-off value is established. Such cut-off value is chosen to match the diagnostic question of interest.

In an embodiment the cut-off is set to result in a specificity of 90%, also preferred the cut-off is set to result in a specificity of 95%, or also preferred the cut-off is set to result in a specificity of 98%.

In an embodiment the cut-off is set to result in a sensitivity of 90%, also preferred the cut-off is set to result in a sensitivity of 95%, or also preferred the cut-off is set to result in a sensitivity of 98%.

In one embodiment values for a protein of the complement factor H family as determined in a control group or a control population are used to establish a reference range. In a preferred embodiment a concentration of a protein of the complement factor H family is considered as elevated if the value determined is above the 90%-percentile of the reference range. In further preferred embodiments a protein concentration of a complement factor H family member is considered as elevated if the value determined is above the 95%-percentile, the 96%-percentile, the 97%-percentile or the 97.5%-percentile of the reference range.

A value above the cut-off value can for example be indicative for an increased clinical benefit in the treatment of the neurodevelopmental, neurological or neuropsychiatric disorders.

In a further preferred embodiment the measurement of a protein of a complement factor H family member(s) is a quantitative measurement. In further embodiments the concentration of a protein of complement factor H family member(s) is correlated to an increased clinical benefit in the treatment of the neurodevelopmental, neurological or neuropsychiatric disorders.

As the skilled artisan will appreciate, any such assessment is made in vitro. The sample (test sample) is discarded afterwards. The sample is solely used for the in vitro diagnostic method of the invention and the material of the sample is not transferred back into the patient's body. Typically, the sample is a body fluid sample, e.g., serum, plasma, or whole blood.

The method according to the present invention is based on a liquid or body fluid sample which is obtained from an individual and on the in vitro determination of protein concentration of Complement factor H family members or a mixture thereof in such sample. An “individual” as used herein refers to a single human.

Preferably the protein concentration of complement factor H family member(s) are specifically determined in vitro from a liquid sample by use of a specific binding agent.

Unless defined otherwise, technical and scientific terms used herein have the same meaning as commonly understood by one of ordinary skill in the art to which this invention belongs.

### Singleton, et al., Dictionary of Microbiology and Molecular Biology, 2nd ed., John Wiley & Sons, New York, N.Y. (1994);

### March, Advanced Organic Chemistry Reactions, Mechanisms and Structure, 4th ed., John Wiley & Sons, New York, N.Y. (1992);

Lewin, B., Genes V, published by Oxford University Press (1994), ISBN 0-19-854287 9;
Kendrew, J., et al., (eds.), The Encyclopedia of Molecular Biology, published by Blackwell Science Ltd. (1994), ISBN 0-632-02182-9; and
Meyers, R. A., (ed.), Molecular Biology and Biotechnology: a Comprehensive Desk Reference, published by VCH Publishers, Inc. (1995), ISBN 1-56081-569 8)
provide one skilled in the art with a general guide to many of the terms used in the present application. All references cited herein are hereby incorporated by reference in their entirety.

Techniques for the detection of protein expression of the respective genes described by this invention include, but are not limited to enzyme linked immunosorbent assay (ELISA).

The practicing of the present invention employs, unless otherwise indicated, conventional techniques of molecular biology (including recombinant techniques), microbiology, cell biology, biochemistry, and immunology, which are within the skill of the art. Such techniques are explained fully in the literature, such as

Sambrook, et al., Molecular Cloning: A Laboratory Manual, second edition, (1989);

### Gait, M. J., (ed.) Oligonucleotide Synthesis (1984);

### Freshney, R. I., (ed.), Animal Cell Culture (1987); Methods in Enzymology (Academic Press, Inc.);

Ausubel, F. M., et al., (eds.), Current Protocols in Molecular Biology (1987) and periodic updates;

### Mullis, et al., (eds.) PCR: The Polymerase Chain Reaction (1994).

The method according to the present invention is based on a liquid or body fluid sample which is obtained from an individual and on the in vitro determination of protein concentration of complement factor H family member(s) in such sample. An “individual” as used herein refers to a single human.

In a preferred embodiment according to the present invention, the protein concentrations of complement factor H family member(s) are determined. In an embodiment, the protein concentration of complement factor H family member(s) are specifically determined in vitro from a sample by use of a specific binding agent.

A specific binding agent is, e.g., a receptor for the complement factor H family member(s), a lectin binding to complement factor H family member(s), an antibody to complement factor H family member(s), peptide-bodies to complement factor H family member(s), bi-specific dual binders or bi-specific antibody formats. A specific binding agent has at least an affinity of 10⁷ l/mol for its corresponding target molecule. The specific binding agent preferably has an affinity of 10⁸ l/mol or also preferred of 10⁹ l/mol for its target molecule.

As the skilled artisan will appreciate the term specific is used to indicate that other biomolecules present in the sample do not significantly bind to the binding agent specific for the complement factor H protein sequence of SEQ ID NO:1 or complement factor H related proteins 1-5 of SEQ ID NO: 2-7. Preferably, the concentration of binding to a biomolecule other than the target molecule results in a binding affinity which is at most only 10% or less, only 5% or less only 2% or less or only 1% or less of the affinity to the target molecule, respectively. A preferred specific binding agent will fulfil both the above minimum criteria for affinity as well as for specificity.

Examples of specific binding agents are peptides, peptide mimetics, aptamers, spiegelmers, darpins, ankyrin repeat proteins, Kunitz type domains, antibodies, single domain antibodies [6] and monovalent fragments of antibodies.

In certain preferred embodiments the specific binding agent is a polypeptide.

In certain preferred embodiments the specific binding agent is an antibody or a monovalent antibody fragment, preferably a monovalent fragment derived from a monoclonal antibody.

The term “antibody” herein is used in the broadest sense and specifically covers monoclonal antibodies, polyclonal antibodies, multi-specific antibodies (e.g. bi-specific antibodies) formed from at least two intact antibodies, and antibody fragments so long as they exhibit the desired biological activity. In certain preferred embodiments the specific binding agent is an antibody or a monovalent antibody fragment, preferably a monovalent fragment derived from a monoclonal antibody.

A specific binding agent preferably is an antibody or a set of antibodies reactive with a member of the complement factor H family or a combination thereof, e.g. complement factor H (SEQ ID NO: 1), complement factor H related protein 1 (SEQ ID NO: 2), complement factor H related protein 2 (SEQ ID NO: 3), complement factor H related protein 3 (SEQ ID NO: 4), complement factor H related protein 4A (SEQ ID NO: 5), complement factor H related protein 4B (SEQ ID NO: 6), complement factor H related protein 5 (SEQ ID NO: 7), either a linear epitope or a conformational epitope.

As the skilled artisan will appreciate now, that protein concentrations of complement factor H family members have been identified as marker which is useful in the assessment of clinical benefit for the treatment of neurodevelopmental, neurological or neuropsychiatric disorders.

For determination of protein concentrations of complement factor H family member(s) the sample obtained from an individual is incubated in vitro with the specific binding agent for complement factor H family member(s) under conditions appropriate for formation of a complex between binding agent and complement factor H family member(s). Such conditions need not be specified, since the skilled artisan without any inventive effort can easily identify such appropriate incubation conditions. The amount of complex between binding agent and complement factor H family member(s) is determined for example in an enzyme-linked immunoassay (ELISA) as described above [7].

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, Practice and theory of enzyme immunoassays, pp. 221-278, and various volumes of Methods in Enzymology, Colowick, S. P., and Caplan, N. O. (eds.), Academic Press), dealing with immunological detection methods, especially volumes 70, 73, 74, 84, 92 and 121.

Using this assay format, samples from patients can be used to assess the clinical benefit for patients by treatment. Results are shown in the example section of this application.

### EXAMPLE

The invention will now be further described in the example below, which is intended as an illustration only and does not limit the scope of the invention.

### Patients and Study Design of Phase 2 GRI Schizophrenia Trial

A Phase II study was performed in adults with schizophrenia with predominantly negative symptoms. The individuals were clinically stable and on stable treatment with second generation antipsychotics. The study was conducted at multiple sites in Brazil, France, Germany, Hungary, Japan, Mexico, Poland, Russia, and the US according to ICH Guidelines for Good Clinical Practice (ClinicalTrials.gov Identifier: NCT00616798). The study protocol was approved by the health authorities of each country and the respective ethic committees of each site. All 323 patients had provided written consent to participate in the study and 224 patients had consented to the collection and use of serum samples for exploratory biomarker analysis. After a screening and a 4-week run-in period, patients were randomized to double-blind adjunctive treatment with placebo, 10 mg, 30 mg, or 60 mg of GRI given once a day for a duration of 8 weeks, followed by 4 week follow-up period. Similar percentages of patients in the four treatment arms completed the study.

### Outcome Measures of Phase 2 GRI Schizophrenia Trial

Patients were assessed at screening, week −2, baseline, weeks 1, 2, 4, 6, 8, 10 and 12. Assessments included the PANSS, Clinical Global Impression—Severity of Illness (CGI-S) and Clinical Global Impression-Global Improvement (CGI-I) for overall psychopathology 56, CGI-S and CGI-I of Negative Symptoms (CGI-S-N, CGI-I-N) for assessment of negative symptoms only 57, and standard examinations of extrapyramidal symptoms (BAS58, SAS59, AIMS60). The primary efficacy variable assessing effects on negative symptoms was the change from baseline in the PANSS negative symptom factor score 64. Secondary efficacy variables were i) proportion of patients showing a clinical response in negative symptom defined as a 20% change from baseline in the PANSS negative symptom factor score and ii) CGI-I-N. Additional analyses included change in PANSS total score, remaining PANSS factor scores 64 CGI-S and CGI-I.

### Preparation of Serum Samples

Serum samples were collected at baseline and at 8 weeks of treatment from patients that had given written consent to the collection and use of serum samples for exploratory biomarker analysis. Samples were collected in a plain tube without EDTA and allowed to stand for approximately 30 min or until clotted at room temperatures. Within 60 min of blood collection samples were transferred into a centrifuge and spun at 1500 g for 10 min at 4° C. (or at room temperature if refrigerated centrifuge was not available). Immediately after centrifugation supernatant (i.e. serum) was transferred into a fresh pre-labelled tube and stored at −70 degree.

### Determination and Results of Concentration of CFH from Patients from Phase 2 GRI Schizophrenia Trial

Baseline serum samples were analyzed on the Luminex based multiplex ELISA Human Discovery Map® v 1.0 provided by Rules Based Medicine (Austin Tex.). Frozen serum-samples from the Phase II study were randomized, blinded and sent on dry ice to Rules Based Medicine for analysis of 189 protein analytes. Protein biomarker measurements were performed in one batch and results were reported in measurement units (eg ng/ml).

Measurement values were reported if they were within the standard calibration curve. Values outside the calibration curve were imputed using the calibration limits. Of the 189 measured protein analytes, 26 reported measurement values below the lowest calibrator for more than 70% of the patient samples. These analytes were removed from analysis. Measurements for 2 related analytes (pro-insulin and insulin) were aggregated by averaging since they showed highly correlated measurement values. After pre-processing 162 protein analytes remained in the analysis data set.

### Demographics, Baseline Psychopathology and Functioning of the Biomarker Subgroup Versus the Total Study Cohort of Phase 2 GRI Schizophrenia Trial

224 out of 323 patients from the Phase 2 GRI Schizophrenia trial had consented to the collection and use of serum samples for exploratory biomarker analysis. The biomarker subgroup is representative for the whole study cohort.

### Identification of Biomarker Candidate for Response Prediction

In the clinical trial as a whole, the primary analysis endpoint was change from baseline in the PANSS negative symptoms factor score [8]. The response variable for this analysis was the change in PANSS Negative Symptom Factor Score between baseline and week 8. For identification of a biomarker for response prediction protein analytes as measured by RBM were analyzed as follows: The only explanatory variable for this analysis was the baseline measurement of an individual protein and the analysis was performed on the pooled 10 mg and 30 mg biomarker population using PP (per protocol) population. For each protein marker candidate the null hypothesis that change in the PANSS is uncorrelated to the protein measurement was tested using RA2 of a robust linear model as test statistic. The null hypothesis was tested for all assayed protein markers in parallel with correction for multiple testing. As TABLE 1 shows an analyte called “Complement factor H” ranked as the best performing marker, however, after adjustment for multiple testing it did not yield statistical significance.

As a secondary endpoint in the clinical trial the Clinical Global Impression (CGI) improvement of negative symptoms between baseline and week 8 evaluation was used. This outcome measure was treated as a binary variable. The positive group consisted of the “Very much improved” and “Much improved” scores, the negative group consisted of “Minimally improved”, “No change”, “Minimally worse”, “Much worse” and “Very much worse”. The analysis was performed on the combined placebo, 10 mg and 30 mg biomarker population using the per protocol (PP) population. Explanatory variables for this analysis are the baseline measurements of all measured proteins, the treatment (placebo or pooled 10 mg/30 mg treatment) and the treatment-protein interaction. A logistic regression model was used as predictor.

Based on this analysis markers were ranked by their ability to discriminate patients with improvement from patients without improvement in CGI on the 10 and 30 mg treatment arms.

The analyte called “Complement factor H” within the RBM panel was identified as the only marker reaching statistical significance after correction for multiple testing with an adjusted p value of 0.006 (see Table 1).

The ability of serum “complement factor H” baseline levels to predict clinical effect was independent of demographic and clinical covariates (including baseline PANSS) and there was no significant difference in “complement factor H” baseline serum concentration between the different treatment arms. “Complement factor H” showed no association with response in the placebo group (PANSS or CGI-I).

Further characterization of the antibodies used in the RBM assay revealed that both the capture and detection antibodies bind within the last 3 short consensus repeats (SCRs) of CFH. This region is highly homologous to complement factor H related protein 1 (CFHR1: SCR18, SCR19 and SCR20 share 100%, 100% and 97% identity with SCR 3, SCR4 and SCR5 of CFHR1, respectively [4].

Human CFH and CFHR1 proteins were isolated from serum and purified. Characterization of both proteins in the RBM assay showed that CFHR1 was recognized with a 10 fold preference for CFHR1 over CFH.

### Antibodies Used for the CFHR1-Specific Assay

For the development of a CFHR1-specific assay the following monoclonal antibodies were used: MAB<CFH/CFHR1>M-L20/3 (provider: Thermo Scientific, cat. no.: GAU 020-03-02) and MAB<CFHR1>M-442127 (provider: R&D-systems, cat.-no.: MAB4247).

### Biotinylation of Monoclonal L20/3 Fab-Fragments, Stoichiometry 1:1.3

Monoclonal mouse IgG (clone: L20/3; provider: Thermo Scientific, cat. no.: GAU 020-03-02) is digested using Papain (3 mU/mg IgG) to produce Fab-fragments. Digested Fc-fragments are eliminated by chromatography on DAE-Sepharose. Purification of Fab by Fcγ-adsorption of remaining Fc followed by Superdex 200 size exclusion.

To a solution of 10 mg/ml L20/3-Fab fragments in 100 mM KPO₄, pH 8.5 50 ul Biotin-N-hydroxysuccinimide (3.6 mg/ml in DMSO) are added per ml. After 45 min at room temperature, the sample is dialysed against 100 mM KPO₄, 150 mM NaCl, pH 7.2 and frozen.

### Ruthenylation of Monoclonal 442127 IgG, Stoichiometry 1:3

To a solution of 5 mg/ml monoclonal mouse IgG (clone 442127, provider: R&D-systems, cat.-no.: MAB4247) in 100 mM KPO4, pH 8.5, 125 ug Ruthenium-(bpy)2-bpyCO-Osu are added. After 75 mM at room temperature, ruthenylation is stopped by addition of 10 mM Lysine. For separation of aggregates appropriate fractions of sample are collected from Superdex 200 size exclusion chromatography.

### Purification of CFHR1 from Human Serum

For use as calibrator material, native CFHR1 was purified from human serum by immunoadsorption using the monoclonal antibody L20/3 specific for CFH and CFHR1 according to following procedure: MAB L20/3<CFH,CFHR1>M-IgG-Spherosil resin is used for purification of CFHR1 from human serum. Prediluted (1:4 in 50 mM TrisHCl, 150 mM NaCl, pH7.5) serum is passed through Sartoclean CA (0.8 μm) and Sartobran P (0.2 um) filter caps. Pretreated serum, loaded on the column is washed (10 mM TrisHCl, 500 mM NaCl, 0.05% Tween20, pH 7.5) followed by Q-sepharose running buffer and eluted in gentle elution buffer (Thermo Scientific) to avoid degradation of analyte and adsorber. For separation of co-bound CFHR1 and CFH, the immunoadsorber-eluate is run over a MonoQ column for size dependent elution. The eluate containing CFHR1 as well as co-purified protein running at different height in SDS-PAGE analysis is further separated for isoelectricity on a MonoS column resulting in fractions of pure CFHR1. Pure CFHR1 is applied as calibrator material for the determination of CFHR1 in the Elecsys-ECLIA test for serum CFHR1 values.

### Measurement of CFHR1 in Human Serum or Plasma Samples Using the ECLIA Immunoassay

ECLIA immunoassay for CFHR1 is developed for the specific measurement of CFHR1 in human serum or plasma samples using the Elecsys® Analyzer.

The following describes the assay procedure for the determination of CFHR1 using the Elecsys® Analyzer.

The Elecsys CFHR1 immunoassay is an electrochemiluminescence immunoassay (ECLIA) that functions via the sandwich principle. There are two antibodies included in the assay—a biotinylated Fab fragment of monoclonal antibody L20/3-Bi (capture antibody) and a ruthenylated monoclonal anti-CFHR1 antibody 442127-Ru (detection antibody)—which form sandwich immunoassay complexes with CFHR1 in the sample. The complexes are then bound to solid-phase streptavidin-coated microparticles. The microparticles are magnetically captured onto the surface of an electrode, and the application of a voltage to the electrode induces chemiluminescent emission, which is measured by a photomultiplier for readouts. Results are determined via an instrument-specific calibration curve.

Samples are automatically diluted 1:400 in Diluent (Roche Diluent MultiAssay for Elecsys Ref. No. 03609987-190). Assay protocol 3 is applied allowing 9 min preincubation of 10 ul diluted sample with 80 ul of ruthenylated <CFHR1>-442127 at 0.5 ug/ml in reaction buffer (Hepes 50 mM, NaCl 150 mM; Thesit/Polidocanol 0.1%; EDTA 1 mM; bovine serum albumin 0.5%) before 80 ul of biotinylated <CFH,CFHR1>-L20/3 at 1.5 ug/ml in reaction buffer plus 30 μl of microparticles are added and incubated for further 9 min. During incubation an antibody analyte antibody sandwich is formed that is bound to the microparticles. Finally the microparticles are transferred to the detection chamber of the Elecsys system for signal generation and readout. For calibration 1:2 dilution series of purified CFHR1 (125 ng/ml, 62.5 ng/ml, 31.25 ng/ml, 15.63 ng/ml, 7.81 ng/ml, 3.9 ng/ml, 1.95 ng/ml, 0 ng/ml) are prepared in MultiAssay Diluent. The equation of the calibration curve was calculated by non-linear least-squares curve-fitting (RCM-Rodbard) and used for converting the signal readout into the corresponding concentration value. The result is multiplied by the pre-dilution factor to get the concentration of the respective sample itself.

Cross-reactivity of the applied antibody sandwich was determined by measuring distinct concentrations of the potentially cross-reacting serum components CFH, CFHL1, CFHR2, CFHR3, CFHR4 and CFHR5. For the applied antibody sandwich no cross-reactivity has been determined for CFHL1, CFHR2, CFHR3, CFHR4, CFHR5 and only minor recognition of CFH of 4.4% was determined.

### Measurement of Serum Samples from Phase 2 GRI Schizophrenia Trial Identifies Natural Cut-Offs Within CHFR1

CFHR1 concentrations measured with the CFHR1 ECLIA immunoassay showed a multi-modal distribution which correlates with the deletion status of the CFHR1 gene.

FIG. 1 shows the distribution of CFHR1 concentrations in the Phase II serum-samples. Three groups were identified:

a group with CFHR1 concentrations below 8 μg/ml, which correlated with the group carrying a homozygous deletion for CFHR1, and thus does not express CFHR1. The measured signal is due to the residual cross-reactivity of the CFHR1 ECLIA immunoassay with CFH.

a group with CFHR1 concentrations above 8 ug/ml and below 28 ug/ml. This group carries a heterozygous deletion for CFHR1.

a group with CHFR1 values above 28 ug/ml. This group carries no deletion for CFHR1. CFHR1 is deleted in about 5-17% of the population, dependent on ethnic origin [5,9]. In Caucasians, about 5% of the population bears a homozygous deletion and about 40% carry a heterozygous deletion.

### Patient Stratification Using CFHR1 Natural Cut-Off

The CFHR1 protein concentrations within each of the three groups shown in FIG. 1 were analyzed for association with treatment response: In patients carrying the homozygous deletion, no CFHR1 can be detected; the residual signal is due to cross-reactivity of the assay with CFH. Within patients carrying the heterozygous deletion of CFHR1 there was no association of CFHR1 protein concentration with treatment response. Within the patient group carrying no deletion of CFHR1 there was a trend where a higher CFHR1 concentration correlated with higher treatment response. This indicates that the response rate is actually mainly predicted by the genetic status of CFHR1, with heterozygous or homozygous deletion of CFHR1 translating into to lower response to GRI.

Consequently, for patient stratification, the natural cut-off of CFHR1 baseline serum concentration identified at 28 ug/ml using the ECLIA immunoassay specific for CFHR1 (FIG. 1) was chosen. Patients with CFHR1 serum values below or equal to 28 ug/ml were stratified as “CFHR1-low”, while patients with CFHR1 serum values above 28 ug/ml were stratified as “CFHR1-high”. Table 2 shows the distribution of CFHR1-high and CFHR1-low patients within the different dose groups

CFHR1-high (N=75) and CFHR1-low (N=75) subgroups were analyzed on the primary endpoint and selected secondary clinical endpoints of the Phase II study and compared to the non-stratified CFHR1 serum-sample PP subgroup (N=150).

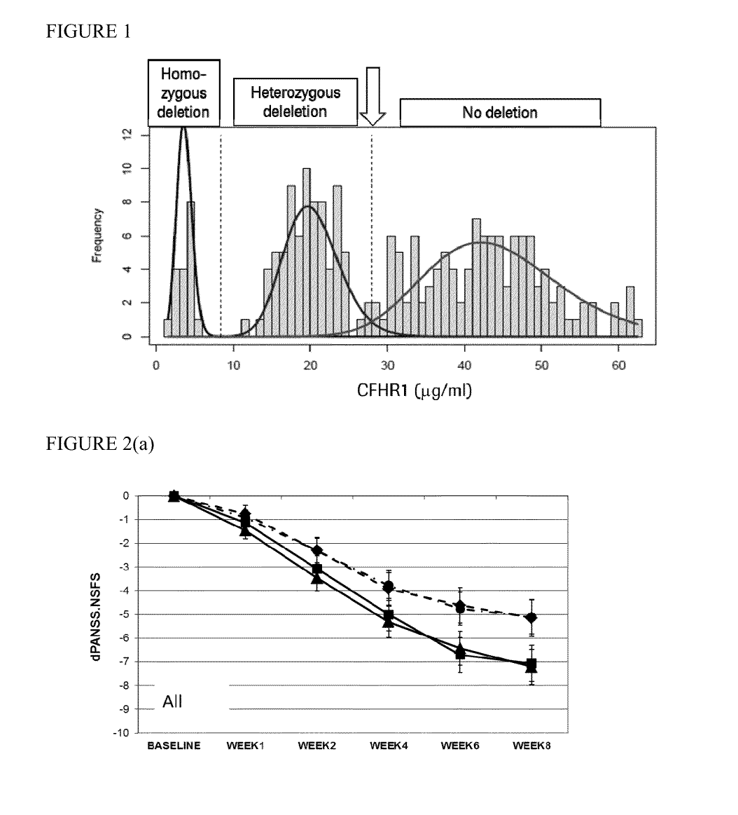

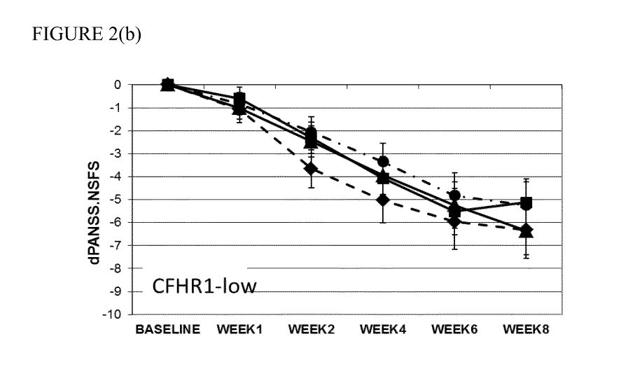

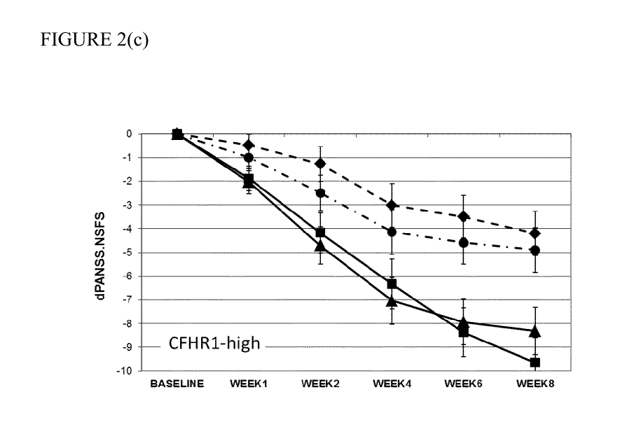

FIG. 2 shows the analysis on the change in PANSS negative symptom factor score (PANSS NFS) from baseline over time. Results for the overall patient cohort are shown in the top panel, for the CFHR1-low patients in the middle panel and for the CFHR1-high patients in the lower panel. For the 10 mg arm the effect size in the CFHR1-high group was −1.01 compared to −0.42 in the non-stratified group, and for the 30 mg arm the effect size in the CFHR1-high group was −0.76 compared to −0.47 in the non-stratified group.

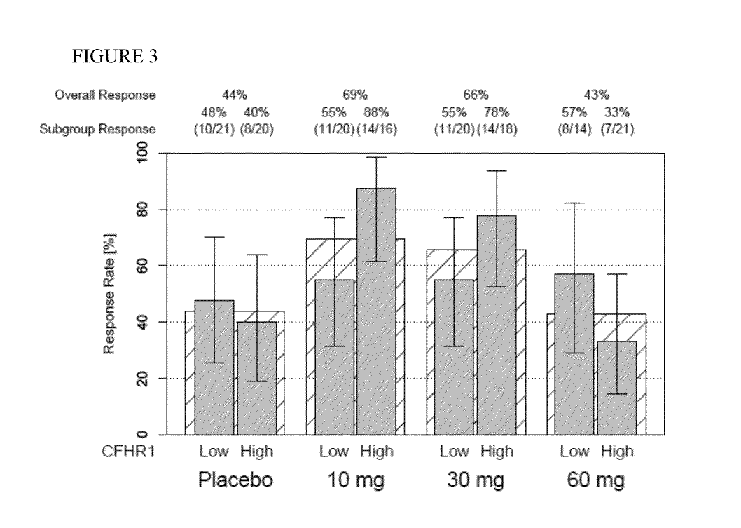

FIG. 3 shows the analysis on the PANSS negative symptom factor score responder rates, which was a secondary clinical endpoint: Patients with at least a 20% decrease from baseline in the PANSS negative symptom factor score after 8 weeks of treatment were defined as responders: The response rate for all four treatment arms in the overall serum subgroup is given as white striped background bars and for the CFHR1-high and CFHR1-low subgroups as grey bars. For the 10 mg arm the response rate in the CFHR1-high group was 88% compared to 69% in the non-stratified group, and for the 30 mg arm the response rate in the CFHR1-high group was 78% compared to 66% in the non-stratified group.

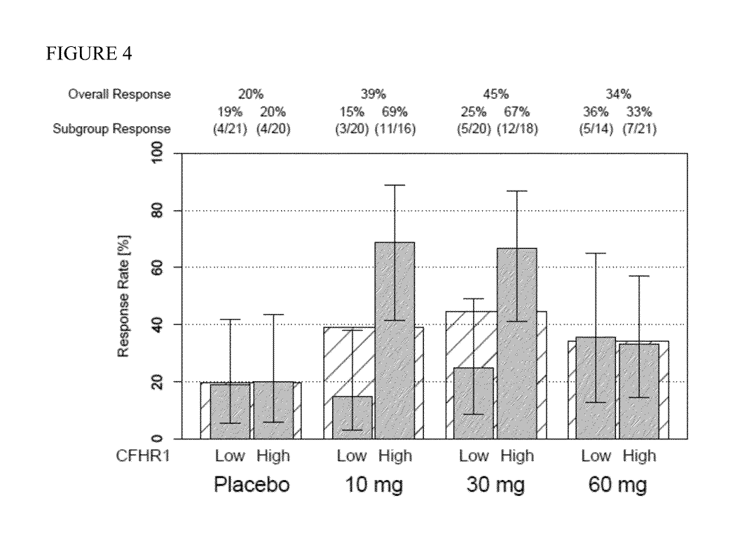

FIG. 4 shows the analysis on another secondary clinical endpoint, the clinical global impression (CGI) of negative symptoms responder rates: For analysis of CGI responder rates, those patients that were “improved” or “very much improved” were defined as responders: The response rate for all four treatment arms in the overall serum subgroup is given as white striped background bars and for the CFHR1-high and CFHR1-low subgroups as grey bars. For the 10 mg arm the response rate in the CFHR1-high group was 69% compared to 39% in the non-stratified group, and for the 30 mg arm the response rate in the CFHR1-high group was 67% compared to 45% in the non-stratified group.

### Antibodies Used for the CFH-Specific Assay

For the development of a CFHR1-specific assay the following monoclonal antibodies were used: MAB<CFH/CFHR1>M-L20/3 (provider: Thermo Scientific, cat. no.: GAU 020-03-02) and MAB<CFH>OX-24 (provider: Thermo Scientific, cat. no.: MA1-70057).

### Ruthenylation of OX24 IgG, Stoichiometry 1:3

To a solution of 5 mg/ml monoclonal mouse IgG (clone OX-24, Thermo Scientific, cat. no.: MA1-70057) 5 mg/ml in 100 mM KPO4, pH 8.5, are added and 125 μg Ruthenium-(bpy)₂-bpyCO-Osu are given to the IgG solution. After 75 min at room temperature, ruthenylation is stopped by addition of 10 mM Lysine. For separation of aggregates appropriate fractions of sample are collected from Superdex 200 size exclusion chromatography.

### Purification of CFH from Human Serum

For use as reference calibrator material, native CFH was purified from human serum by immunoadsorption using the monoclonal antibody L20/3 specific for CFH and CFHR1 according to following procedure: MAB L20/3<CFH,CFHR1>M-IgG-Spherosil resin is used for purification. Prediluted (1:4 in 50 mM TrisHCl, 150 mM NaCl, pH7.5) serum is passed through Sartoclean CA (0.8 μm) and Sartobran P (0.2 μm) filter caps. Pretreated serum, loaded on the column is washed (10 mM TrisHCl, 500 mM NaCl, 0.05% Tween20, pH 7.5) followed by Q-sepharose running buffer and eluted in gentle elution buffer (Thermo Scientific) to avoid degradation of analyte and adsorber. For exclusion of co-bound CFHR1, the eluate from immunoadsorber is run over a MonoQ column for size dependent elution. Pure CFH is applied as reference calibrator material for the determination of CFH in the Elecsys-ECLIA test for serum CFH values.

### Measurement of CFH in Human Serum or Plasma Samples Using the ECLIA Immunoassay

ECLIA immunoassay for CFH is developed for the specific measurement of CFH in human serum or plasma samples using the Elecsys® Analyzer.

The following describes the assay procedure for the determination of CFH using the Elecsys® Analyzer.

The Elecsys CFH immunoassay is an electrochemiluminescence immunoassay (ECLIA) that functions via the sandwich principle. There are two antibodies included in the assay—a biotinylated Fab fragment of monoclonal anti-CFH antibody L20/3-Bi (capture antibody) and a ruthenylated monoclonal anti-CFH antibody OX-24-Bi Ru (detection antibody)—which form sandwich immunoassay complexes with CFH in the sample. The complexes are then bound to solid-phase streptavidin-coated microparticles. The microparticles are magnetically captured onto the surface of an electrode, and the application of a voltage to the electrode induces chemiluminescent emission, which is measured by a photomultiplier for readouts. Results are determined via an instrument-specific calibration curve which is generated by 2-point calibration and a master curve provided via the reagent barcodecurve. The total time required to perform the assay is 18 minutes.

Samples are automatically diluted 1:400 in MultiAssay Diluent for Elecsys (Roche 03609987-190). Following the applied assay protocol 2, 80 μl of biotinylated <CFH,CFHR1>-L20/3 at 1.5 μg/ml and 80 μl of ruthenylated <CFH>-OX24 at 1.5 μg/ml both in reaction buffer (Hepes 50 mM, NaCl 150 mM; Thesit/Polidocanol 0.1%; EDTA 1 mM; bovine serum albumin 0.5%) are incubated with 10 μl of sample. During incubation an antibody analyte antibody sandwich is formed that is bound to the microparticles. Finally the microparticles are transferred to the detection chamber of the Elecsys system for signal generation and readout. For calibration 1:4 dilution series of purified CFH (1.25 μg/ml, 312.5 ng/ml, 78.1 ng/ml, 19.5 ng/ml, 0 ng/ml) (500 μg/ml, 125 μg/ml, 31.25 μg/ml, 7.81 μg/ml, 0 μg/ml)—are prepared in MultiAssay Diluent. The equation of the calibration curve was is calculated by non-linear least-squares curve-fitting (RCM-Rodbard) and used for converting the signal readout into the corresponding concentration value. The result is multiplied by the pre-dilution factor to get the concentration of the respective sample itself.

Cross-reactivity of the applied antibody sandwich was determined by measuring distinct concentrations of the potentially cross-reacting serum components. For the applied antibody sandwich cross-reactivity of 2.9% has been determined for CFHR1

### Measurement of CFHR1/CFH Ratio Measured in Serum Samples from Phase 2 GRI Schizophrenia Trial Confirms Natural Cut-Offs

CFHR1 concentration and CFH concentration are measured independently with the CFHR1 and CFH ECLIA immunoassay and allowed calculation of the CFHR1/CFH ratio. Similar to CFHR1 concentration alone as shown in FIG. 1, the CFHR1/CFH ratio showed a multi-modal distribution which correlates with the deletion status of the CFHR1 gene.

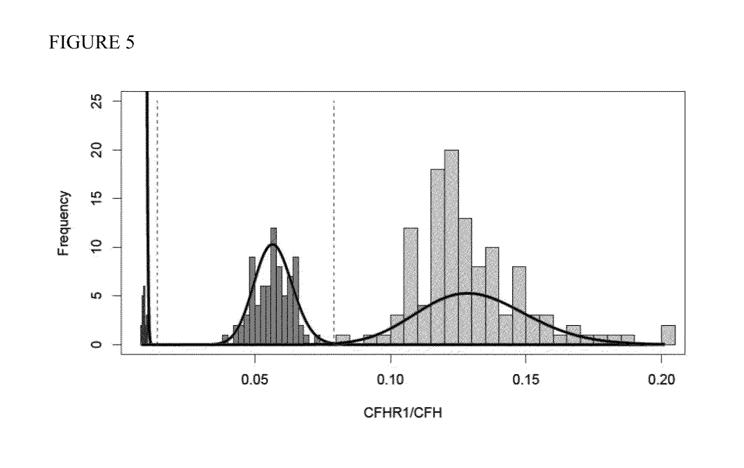

FIG. 5 shows the distribution of the CFHR1/CFH ratio in the Phase II serum-samples. Three groups were identified:

a group with a CFHR1/CFH ratio below 0.01, which correlated with the group carrying a homozygous deletion for CFHR1, and thus does not express CFHR1. The residual signal measured is due to the low cross-reactivity of the CFHR1 ECLIA immunoassay with CFH.

a group with a CFHR1/CFH ratio above 0.01 and below 0.08. This group carries a heterozygous deletion for CFHR1.

a group with a CHFR1/CFH ratio above 0.08. This group carries no deletion for CFHR1.

### Patient Stratification Using the CFHR1/CFH Ratio

The CFHR1/CFH ratio within each of the three groups as shown in FIG. 5 were analyzed for association with treatment response. For this purpose a CFHR1/CFH ratio below 0.01 was called low (“L”), a CFHR1/CFH ratio above 0.01 and above 0.08 was called medium (“M”) and a CFHR1/CFH ratio above 0.08 was called high (“H”).

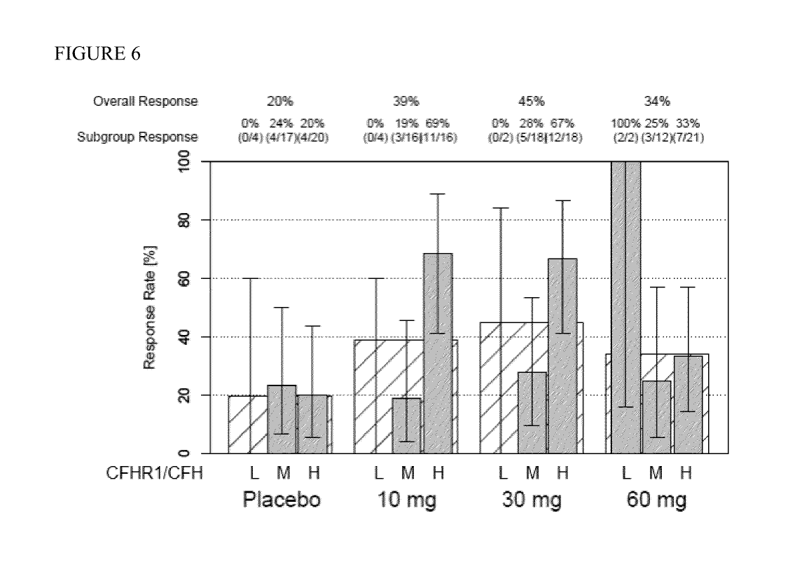

FIG. 6 shows the analysis on the clinical global impression (CGI) of negative symptoms responder rates: For analysis of CGI responder rates, those patients that were “improved” or “very much improved” were defined as responders: The response rate for all four treatment arms in the overall serum subgroup is given as white striped background bars and for the CFHR1/CFH ratio subgroups L, M and H as grey bars. For the L group the response rate was 0% for the Placebo arm and for the 10 mg and 30 mg arm, while in the 60 mg arm the response was 100%. The L group consisted of a small sample size and thus results need to be interpreted with some caution. For the M subgroup the response rate with 24% in the Placebo arm and with 25% in the 60 mg arm were comparable to the non-stratified group (shaded bar), being 20% for Placebo and 34% for 60 mg arm. However, in the 10 mg and 30 mg arms the M subgroup showed lower response rates, 19% (for 10 mg) and 28% (for 30 mg) compared to that of the unstratified group with 39% (for 10 mg) and 45% (for 30 mg). The H subgroup showed a response rate of 20% for the placebo arm and 33% for the 60 mg arm which is comparable to the response rate of the non-stratified group, being 20% (for placebo) and 34% (for 60 mg). In both treatment arms, the 10 mg arm and the 30 mg arm, the H subgroup showed increased treatment response rates: a response rate of 69% in the H subgroup compared to 39% in the non-stratified group for the 10 mg arm, and a response rate of 67% in the H subgroup compared to 45% in the non-stratified group for the 30 mg arm.

### Preparation of DNA Samples

DNA samples were collected from patients that had given written consent to the collection and use of DNA samples for exploratory biomarker analysis (170 of 320 patients). DNA samples were collected as whole blood, collected in a 9 ml EDTA tubes and stored at −20° C. DNA samples were double coded and de-anonymized. From a sample of 50-200 uL whole blood, genomic DNA was extracted using the Roche MagNA Pure 96 LC DNA Isolation System. The principle of DNA isolation is based on magnetic bead technology. Briefly, the samples are first lysed by incubation with a buffer containing chaotropic salts and Proteinase K. Magnetic Glass Particles (MGP) are then added and the DNA binds to their surfaces. Unbound substances are removed by several washing steps and the purified DNA is eluted. The resultant DNA was normalized to a concentration of 5 ng/ul using spectrophotometric quantification.

### Assay for Determination of CFHR1 Copy Number

A quantitative real-time PCR assay was set up to determine the copy number at the genomic location at chromosome 1:196796257-196796381 (coordinates according to according to Genome Build 37, Assembly GRCh37). Oligonucleotide sequences are given in Seq. Id. No. 8, Seq. Id. No. 9 and Seq. Id. No. 10. The fluorescent probe was modified at the 5′ terminus with 5′-Fluorescein and at the 3′ terminus with the 3′ terminal BlackHole™ Dark Quencher-1.

This CFHR1 copy number assay was set in reference to assays labeled with a separable fluorescent reporter that detect a sequence known to exist in two copies in a diploid genome. As reference assays the TaqMan Copy Number Reference Assays for RNase P H1 RNA and hTERT genes (Life Technologies, Carlsbad, Calif.), respectively were employed. Results were considered valid if the resultant CFHR1 copy number from independent duplex setups using either RNaseP and hTERT reference assays matched.

The PCR reaction contained PCR oligonucleotides at a final concentration of 0.9 uM and the probe oligonucleotide at a final concentration of 0.25 uM Real-time PCR reactions were conducted using a LightCyclerR 480 II real-time PCR system and the following cycling parameters: 10 min 95° C., 40×[15 sec 95° C., 1 min 60° C.], 1 min 40° C. Employing “Absolute Quantitation/2nd Derivative Max” instrument settings, the threshold cycle (C_{T}) values were calculated. Using comparative relative quantitation analysis ΔΔCT [10] the copy number of the CFHR1 target sequence was calculated.

As an external reference for the copy number of CFHR1, DNA from the cell line NA07000 (Coriell Institute for Medical Research, Camden, N.J.) was used, which is described to harbor two copies at a location within the CFHR3-1 deletion [11]. The assay was tested on multiple cell lines that are described to harbor one or zero copies at the CFHR3-CFHR1 locus [11] and the specificity of the assay was confirmed.

### Assay for Determination of Rs7542235

DNA samples were genotypes using the Human 1M-Duo Beadchip (v.3) from Illumina according to the manufactures procedure. Samples were then analyzed for SNP rs 7542235.

### Correlation of CFHR1 Copy Numbers to Single Nucleotide Polymorphism rs7542235

The Single Nucleotide Polymorphism (SNP) rs7542235, located at genomic position Chr 1: 196823613 (Genome Build 37, Assembly GRCh37) has been described as a proxy for the common CFHR1-CFHR3 deletion [12]. This association was shown to be very high for individuals with european ancestry ethnicity (r2=1.00) but less tight for other ethnic groups [5, 9].

TABLE 3 shows the correlation of the customized copy number variation assay with the SNP rs7542235 allele: consistent with the literature the major A/A allele tags two CFHR1 copies (denoted CFHR1+/+), the allele A/G tags one CFHR1 copy (denoted CFHR1+/−) and the minor allele G/G tags zero CFHR1 copies (denoted CFHR1−/−).

Noteworthy, both samples with CFHR1−/− and rs7542235 A/A allele are of black, non-hispanic ethnicity and four out of five patients with CFHR1+/− and rs7542235 A/A allele are of non-European ethnicity.

### Correlation of CFHR1 Copy Numbers with CFHR1 Protein Concentration

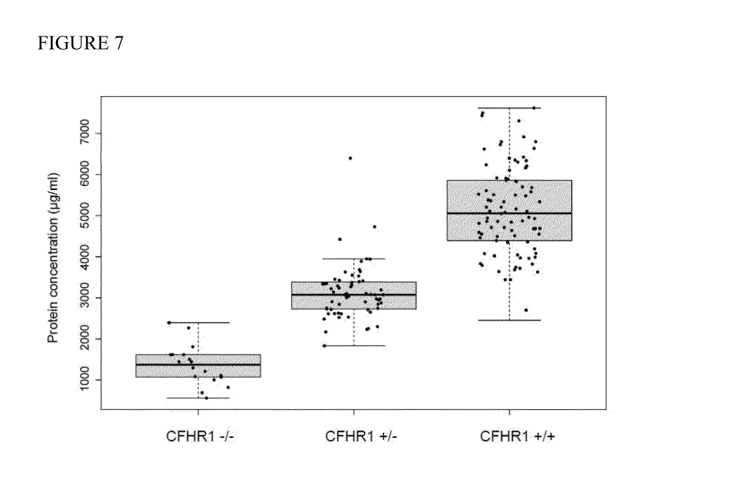

FIG. 7 shows the box plot of the genetic CFHR1 status versus protein concentration measured using the RBM assay (so mainly detecting CFHR1): CFHR1+/+ genetic status correlates with high CFHR1 serum concentration, CFHR1+/− genetic status correlates with lower CFHR1 serum concentration and CFHR1−/− genetic status correlates with the lowest protein signals (caused by crossreactivity of the assay with CFH).

### Correlation of SNP Rs 7542235 to CFHR1 Protein Concentration

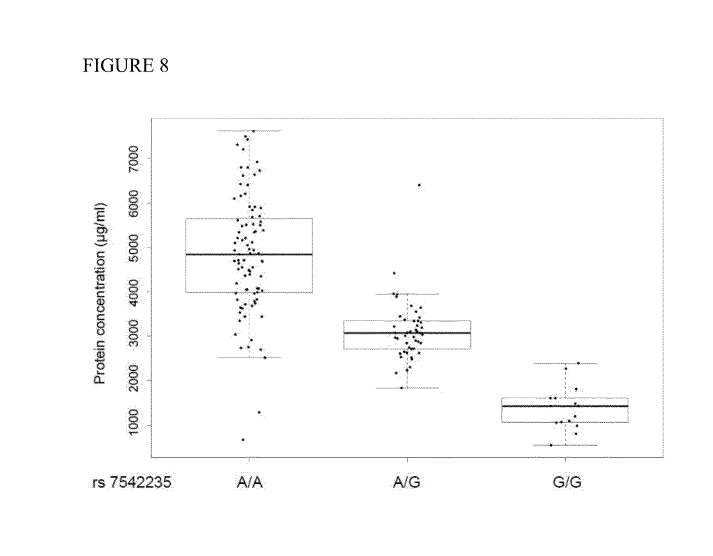

FIG. 8 shows the box plot of the allelic status of the SNP rs 7542235 versus protein concentration measured using the RBM assay (so mainly detecting CFHR1): rs7542235 allele A/A correlates with high CFHR1 serum concentration, rs7542235 allele A/G correlates with lower CFHR1 serum concentration and rs7542235 allele A/A correlates with the lowest protein signals (caused by crossreactivity of the assay with CFH).

### Patient Stratification Using CFHR1 Genetic Analysis

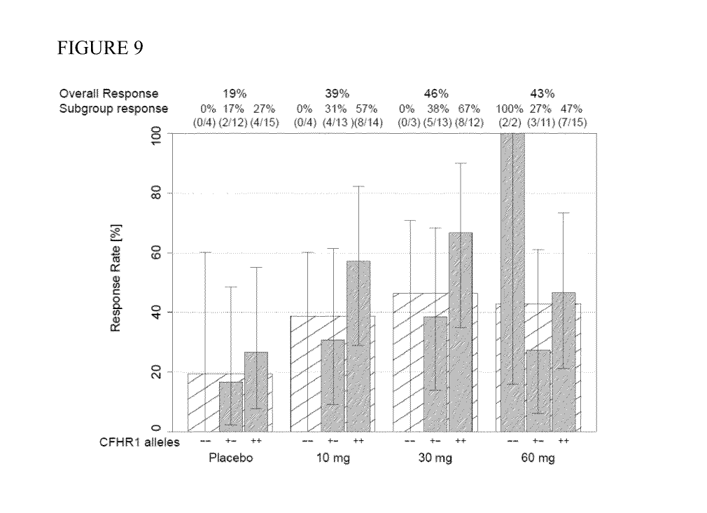

FIG. 9 shows the analysis on the clinical global impression (CGI) of negative

symptoms responder rates: For analysis of CGI responder rates, those patients that were “improved” or “very much improved” were defined as responders: The response rate for those patients who had consented for a DNA sample is given as white striped background bars and for the CFHR1-genetic status as grey bars. For the 10 mg arm the response rate in the CFHR1+/+ group was 57% compared to 39% in the non-stratified group, and for the 30 mg arm the response rate in the CFHR1+/+ group was 67% compared to 46% in the non-stratified group.

TABLE 1
Highest ranking biomarker candidates for predicting PANSS and
CGI-I negative symptom scores at week 8 in patients treated with
10 or 30 mg GRI

| | PANSS | | | CGI-I | | |
|---|---|---|---|---|---|---|
| | Standard- | Un- | | | Un- | |
| | ized | adjusted | Adjusted | | adjusted | Adjusted |
| | Slope | P | P | AUC | P | P |
| “CFH” | −1.33 | 0.004 | 0.17 | 0.75 | 0.0003 | 0.006 |
| CCL23 | 0.54 | >0.2 | >0.2 | 0.70 | 0.004 | 0.11 |
| (MPIF-1) | | | | | | |
| FAS | 0.45 | >0.2 | >0.2 | 0.69 | 0.005 | >0.2 |
| FABP | 0.55 | 0.18 | >0.2 | 0.68 | 0.009 | >0.2 |
| Leptin | −1.30 | 0.03 | >0.2 | 0.63 | 0.06 | >0.2 |
| CXCL9 | 0.51 | >0.2 | >0.2 | 0.67 | 0.01 | >0.2 |
| MMP-3 | 1.06 | 0.03 | >0.2 | 0.59 | 0.19 | >0.2 |
| Apolipo- | −0.91 | 0.03 | >0.2 | 0.58 | >0.2 | >0.2 |
| protein | | | | | | |
| A2 | | | | | | |
| IL-11 | −1.11 | 0.03 | >0.2 | 0.46 | >0.2 | >0.2 |

TABLE 2
Distribution of CFHR1-high and CFHR1-low patients in different
dose arms using natural CFHR1 cut-off.

| | Overall | CFHR1-low | CFHR1-high |
|---|---|---|---|
| | N | N (%) | N (%) |
| All dose arms | 150 | 75 (50%) | 75 (50%) |
| Placebo | 41 | 21 (51%) | 20 (51%) |
| 10 mg | 36 | 20 (56%) | 16 (44%) |
| 30 mg | 38 | 20 (53%) | 18 (47%) |
| 60 mg | 35 | 14 (40%) | 21 (60%) |

CFHR1 low are patients with baseline CFHR1 serum concentration ≦28 μg/ml,
CFHR1 high group are patients with baseline CFHR1 serum concentration >28 μg/ml.
CFHR1 was determined using the ECLIA CFHR1 assay

TABLE 3
Correlation of SNP rs7542235 alleles to CFHR1 genetic status
determined for DNA samples from the Phase 2 GRI Schizophrenia trial

| | rs7542235 A/A | rs7542235 A/G | rs7542235 G/G |
|---|---|---|---|
| CFHR1 −/− | 2 | 0 | 15 |
| CFHR1 +/− | 5 | 52 | 0 |
| CFHR1 +/+ | 88 | 0 | 0 |

### REFERENCES

[1] Walport, M. J. N. Engl. J. Med., 344, 1058-1066
[2] Isohanni, Eur. Arch. Psychiatry Clin Neurosci., 2000, 250(6), 311-9
[3] Marenco, S, Dev. Psychopathol. 2000, 12(3), 501-27
[4] Jozsi, M and Zipfel, P F Trend in Immunology 2008, 29(8), 380-387
[5] Zhao J. et al, PLoS Genetics, May 2011, Vol. 7(5), e1002079,
[6] Hey, T., et al., Trends Biotechnol. 23 (2005) 514-522;
[7] Tietz fundamentals of clinical chemistry, C. A. Burtis and E. R. Ashwood, 5^{th} edition, 2000, pages 177-194, Principles of Immunochemical Techniques;
[8] Marder S R, et al., J Clin Psychiatry. 1997; 58:538-546.
[9] Hagemann et al, Ann Med. 2006, 38 (8), 592-604;
[10] Livak & Schmittgen, Methods. 2001 December; 25(4):402-8.);
[11] Conrad, D F et al., Nature, 2010, 464(7289):704-12.
[12] Raychaudhuri et al. (2011), Nat. Genet. 43, 1232);

1. An in vitro method of predicting whether a patient, having a neurodevelopmental, neurological or neuropsychiatric disorder, will derive a clinical benefit if treated with a glycine reuptake inhibitor (GRI), comprising:
i) assaying and determining in vitro the protein concentration of one, two, three, four five or six members of the complement factor H family or a mixture or a combination thereof in a sample of a human patient having a neurodevelopmental, neurological or neuropsychiatric disorder,
ii) comparing the protein concentration determined in step i) to a value representative of the protein concentration of one, two three, four, five or six members of complement factor H family in a patient population having neurodevelopmental, neurological or neuropsychiatric disorders that did not derive clinical benefit from the treatment with GRI, and
iii) predicting that said human patient will derive clinical benefit from treatment with GRI when a higher protein concentration of one, two three, four five or six members from complement factor H family is detected in the sample of said patient having neurodevelopmental, neurological or neuropsychiatric disorders in comparison to the value representative level of the population of patients having said disorders that did not derive clinical benefit from the treatment,
wherein the complement factor H family member(s) is an individual member of complement factor H family containing complement factor H(CFH), complement factor H related protein 1 (CFHR1), complement factor H related protein 2 (CFHR2), complement factor H related protein 3 (CFHR3), complement factor H related protein 4A (CFHR4A), complement factor H related protein 4B (CFHR4B), and complement factor H related protein 5 (CFHR5) or a mixture or a combination thereof,
wherein further the neurodevelopmental, neurological or neuropsychiatric disorders comprise negative or positive symptoms of schizophrenia, bipolar disorder, substance dependence, autism and compulsive disorders.

2. The in vitro method of claim 1 wherein the GRI compound is [4-(3-fluoro-5-trifluormethyl-pyridin-2-yl)-piperazin-1-yl]-[5-methanesulfonyl-2-[[(2S)-1,1,1-trifluoropropan-2-yl]oxy]phenyl]methanone.

3. The in vitro method of claim 1 wherein the complement factor H family members are a mixture of complement factor H and complement factor H related protein 1.

4. The in vitro method of claim 1 wherein the complement factor H family member is complement factor H related protein 1.

5. The in vitro method of claim 1 wherein the protein concentration of individual members of the complement factor H family or a mixture or a combination thereof are determined by ELISA based technology.

6. The in vitro method of claim 1 wherein the protein concentration of individual members of the complement factor H family or a mixture or a combination thereof are determined by measuring genetic variants of complement factor H family members.

7. The in vitro method of claim 1 wherein the protein concentration of CFHR1 is determined by measuring of genetic variants of CFHR1, either via measurement of copy number variations of CFHR1 or by measurement of a SNP as a proxy for the deletion.

8. The in vitro method of claim 1 wherein the patient is affected with schizoaffective disorder.
